(19) United States
(12) Patent Application Publication
(10) Pub. No.: US 2005/0004172 A1
(43) Pub. Date: Jan. 6, 2005

(54) Solid state forms of 5-(difluoro-methoxy)-2-((3,4-dimethoxy-2-pyridinyl)-methyl) sulfinyl]-1H-benzimidazole sodium aquo complexes

(75) Inventors: Darko Filic, Zagreb (HR); Nada K. Hulita, Susedgrad (HR); Aleksandar Danilovski, Rijeka (HR); Zvonimir Siljkovic, Zagreb (HR); Helena Ceric, Zagreb (HR); Miljenko Dumic, Zagreb (HR); Miroslav Zegarac, Prelog (HR)

Correspondence Address: DARBY & DARBY P.C., P. O. BOX 5257, NEW YORK, NY, 10150-5257, US

(73) Assignee: Pliva-Istrazivacki Institut d.o.o., Zagreb (HR)

(21) Appl. No.: 10/847,900

(22) Filed: May 17, 2004

Related U.S. Application Data
(60) Provisional application No. 60/472,034, filed on May 19, 2003.

(51) Int. Cl. A61K 31/4439; C 07D 4 3/02

(52) U.S. Cl. 514/338; 23/295 R; 546/273.7

(57) ABSTRACT

The present disclosure relates to new solid-state forms of 5-(difluoromethoxy)-2-[[(3,4-dimethoxy-2-pyridinyl)methyl]sulfinyl]-1H-benzimidazole sodium aqua complexes, and to processes for their preparation. The disclosure is also directed to pharmaceutical compositions containing the solid-state forms, and the methods of treatment using the solid-state forms.

Under 35 U.S.C. § 119(e), this application claims the benefit of prior U.S. Provisional Application No. 60/472,034, filed May 19, 2003, which is incorporated herein by reference in its entirety.

### FIELD OF THE INVENTION

The present disclosure relates to new solid-state forms of 5-(difluoromethoxy)-2-[[(3,4-dimethoxy-2-pyridinyl)methyl]sulfinyl]-1H-benzimidazole sodium aqua complexes, and to processes for their preparation. The disclosure is also directed to pharmaceutical compositions containing these solid-state forms, and to methods of treatment using the solid-state forms.

### BACKGROUND OF THE INVENTION

Pantoprazole is an irreversible proton pump inhibitor which has the chemical structure:

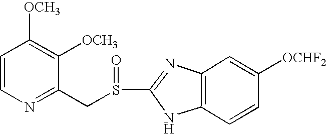

Pantoprazole is used, as an active pharmaceutical ingredient, in the treatment of gastric ulcers, usually in the form of its sodium salt. This was described in European Patent Application No. EP-A-0166287.

It is known that pantoprazole sodium salt can exist as a monohydrate (European Patent No. 0533790) or as a sesquihydrate (European Patent No. 0589981).

### SUMMARY OF THE INVENTION

The present disclosure is directed, in part, to new solid-state forms of 5-(difluoromethoxy)-2-[[(3,4-dimethoxy-2-pyridinyl)methyl]sulfinyl]-1H-benzimidazole sodium aqua complexes.

In one embodiment, the solid-state form is an organic solvent free hexacoordinated octahedral sodium aqua complex of pantoprazole, solid-state Form N.

In another embodiment, the solid-state form is an acetone solvate hexacoordinated octahedral sodium aqua complex of pantoprazole, solid-state Form A1.

In another embodiment, the solid-state form is an acetone solvate pentacoordinated square pyramidal sodium aqua complex of pantoprazole, solid-state Form A2.

In another embodiment, the solid-state form is an acetone solvate sodium aqua complex of pantoprazole, solid-state Form A3.

In another embodiment, the solid-state form is an acetone solvate sodium aqua complex of pantoprazole, solid-state Form A4.

In another embodiment, the solid-state form is a methyl acetate hexacoordinated octahedral sodium aqua complex of pantoprazole, solid-state Form B1.

In another embodiment, the solid-state form is a methyl acetate sodium aqua complex of pantoprazole, solid-state Form B2.

In another embodiment, the solid-state form is a methyl acetate sodium aqua complex of pantoprazole, solid-state Form B3.

In another embodiment, the solid-state form is a methyl ethyl ketone solvate hexacoordinated octahedral sodium aqua complex of pantoprazole, solid-state Form C1.

In another embodiment, the solid-state form is a methyl ethyl ketone solvate sodium aqua complex of pantoprazole, solid-state Form C2.

In another embodiment, the solid-state form is a diethyl ketone solvate hexacoordinated octahedral sodium aqua complex of pantoprazole, solid-state Form D1.

In another embodiment, the solid-state form is a desolvated sodium aqua complex of pantoprazole, solid-state Form E1.

The present disclosure is also directed to processes for preparing the new solid-state Forms N, A1, A2, A3, A4, B1, B2, B3, C1, C2, D1, and E1.

A further embodiment is the use of the solid-state octahedral sodium aqua complexes of pantoprazole of the present invention as raw materials for the preparation of (i) the monohydrate and sesquihydrate forms of pantoprazole sodium, (ii) the pantoprazole hexacoordinated octahedral sodium aqua complexes and pantoprazole pentacoordinated square pyramidal aqua complexes of the present invention, and (iii) other pharmaceutically acceptable pantoprazole salts, such as, but not limited to, the magnesium salt of pantoprazole.

Yet another embodiment of this disclosure is directed to pharmaceutical compositions containing one or more of the solid-state forms of sodium aqua complexes of pantoprazole of the present invention.

Further embodiments provide methods for inhibiting gastric acid secretion, protecting the stomach and intestines, and treating gastric ulcers by administering to a patient in need of such treatment a therapeutically effective amount of one or more of the solid-state forms of sodium aqua complexes of pantoprazole of the present invention, or a composition containing a therapeutically effective amount of one or more of these solid-state forms.

### BRIEF DESCRIPTION OF THE DRAWINGS

FIG. 1 is a crystal packing diagram of the new solid-state solvent free pantoprazole hexacoordinated octahedral sodium aqua complex, Form N.

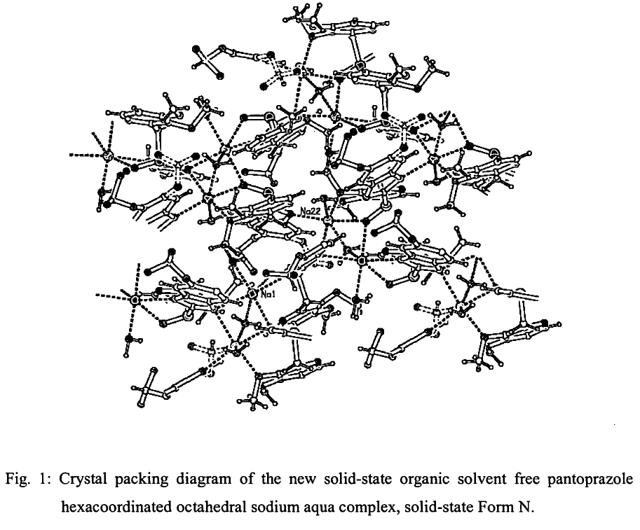

FIG. 2 is a crystal packing diagram of the new solid-state acetone solvate form of pantoprazole hexacoordinated octahedral sodium aqua complex, Form A1.

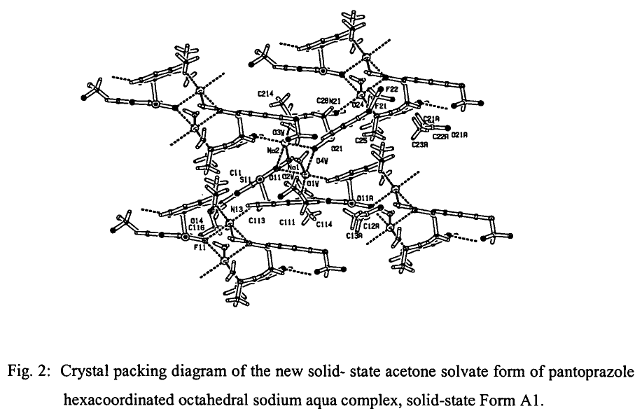

FIG. 3 is a crystal packing diagram of the new solid-state acetone solvate form of pantoprazole pentacoordinated square pyramidal sodium aqua complex, Form A2.

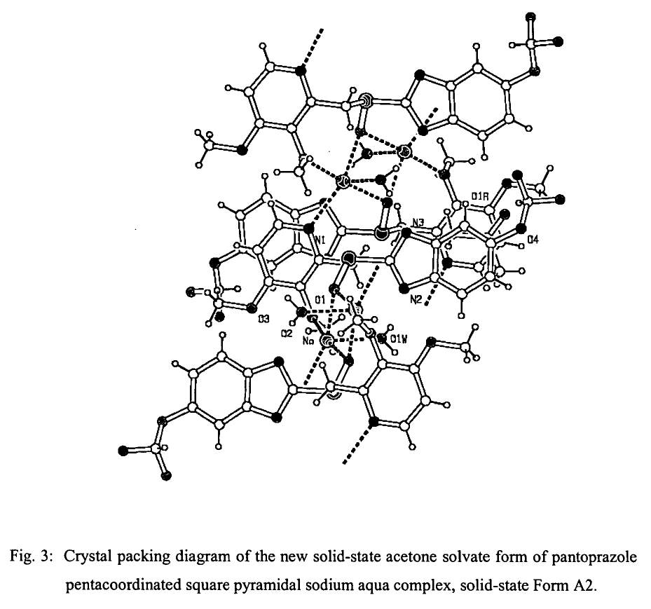

FIG. 4 is a crystal packing diagram of the new solid-state methyl acetate solvate form of pantoprazole hexacoordinated octahedral sodium aqua complex, Form B 1.

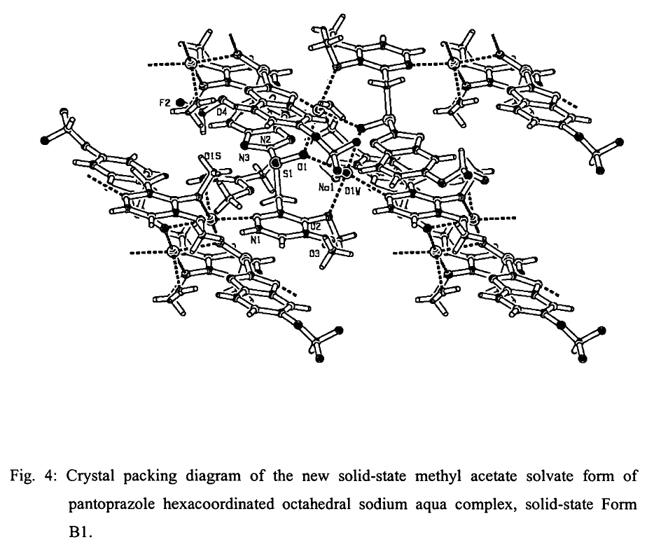

FIG. 5 is a crystal packing diagram of the new solid-state methyl ethyl ketone solvate form of pantoprazole hexacoordinated octahedral sodium aqua complex, Form C1.

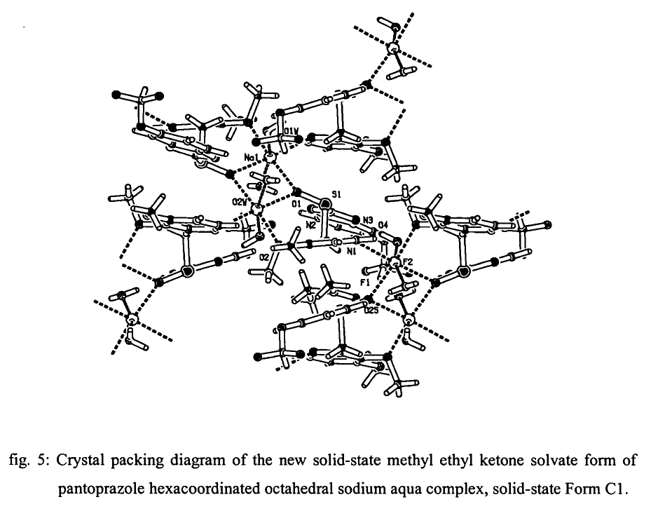

FIG. 6 is a crystal packing diagram of the new solid-state diethyl ketone solvate form of pantoprazole hexacoordinated octahedral sodium aqua complex, Form D1.

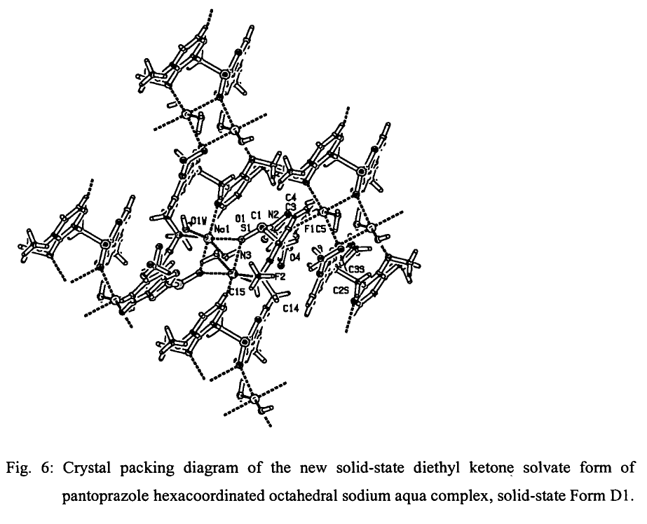

### DETAILED DESCRIPTION OF THE INVENTION

One object of this disclosure is to provide new solid-state forms of pantoprazole sodium aqua complexes.

Solid-State Form N

The new solid-state Form N organic solvent free hexacoordinated octahedral sodium aqua complex of pantoprazole, prepared according to the process of the present invention, has the form of a flowable crystalline powder having the property of flowability, i.e. it is obtained in a “free-flow” form which is not statically chargeable.

Single crystals of the new solid-state Form N were prepared according to the process set forth herein, and single crystal x-ray diffraction data collected using a Bruker Nonius FR591/KappaCCD diffractometer using CuKα radiation. Basic crystallographic data for the new solid-state Form N are represented in Table 1.

TABLE 1
Basic crystallographic data for the new solid-state
Form N organic solvent free hexacoordinated
octahedral sodium aqua complex of pantoprazole.
Form N

| | |
|---|---|
| Empirical formula | [Na₂(C₁₆H₁₄F₂N₃O₄S)₂(OH₂)₃] |
| Formula weight | 863.74 |
| Temperature | 100(2) K |
| Crystal size | 0.05 × 0.15 × 0.70 mm |
| Crystal system, space | Orthorhombic, P bca |
| group | |
| Unit cell dimensions | a = 17.10(2) Å |
| | b = 13.49(1) Å |
| | c = 33.15(2) Å |
| | α = β = γ = 90° |
| Volume | 7647.5(1) Å³ |
| Z | 8 |
| Calculated density | 1.50 gcm⁻³ |

The new solid-state Form N has a characteristic x-ray powder pattern obtained by x-ray diffraction on a powder sample of the organic solvent free Form N.

The new solid-state Form N has characteristic x-ray powder diffraction peaks designated by “2Θ” and expressed in degrees, as follows: 5.3±0.2°, 13.1±0.2°, 16.9±0.2°, 20.5±0.2°, 21.6±0.2° and 25.1±0.2°. X-ray powder patterns were collected using a Philips X'PertPRO powder diffractometer using CuKα radiation.

The new solid-state Form N can be obtained by crystallization from solutions of pantoprazole sodium salt in organic solvents and water. A process for the preparation of the new solid-state Form N organic solvent free hexacoordinated octahedral sodium aqua complex of pantoprazole comprises:
(i) suspending pantoprazole sodium salt in an organic solvent or mixture of organic solvents;
(ii) dissolving the pantoprazole sodium salt in the organic solvent or mixture of organic solvents;
(iii) optionally filtering the solution of pantoprazole sodium salt and organic solvent or mixture of organic solvents;
(iv) adding water;
(v) crystallizing the new solid-state Form N solvent free hexacoordinated octahedral sodium aqua complex of pantoprazole;
(vi) isolating the crystals thus obtained; and
(vii) drying the crystals.

Organic solvents suitable in the process include, but are not limited to, aliphatic esters, such as ethyl acetate, propyl acetate, isopropyl acetate, butyl acetate, sec-butyl acetate and tert-butyl acetate, and mixtures thereof.

For example, for the preparation of the new solid-state Form N, the organic solvent used may be an aliphatic ester chosen from, but not limited to, ethyl acetate and butyl acetate, or mixtures thereof.

In one embodiment of step (ii) of the process for the preparation of the new solid-state Form N, the suspension of pantoprazole sodium salt and organic solvent is heated to a temperature of from about 30° C. to about reflux for a time sufficient to obtain clear solution.

In one embodiment of step (iv) of the process for the preparation of the new solid-state Form N, water can be added in an amount of about 0.1% to about 5% by volume of the organic solvent or solvents, for example, in an amount of about 2.5% by volume of the organic solvent or solvents.

In one embodiment of the step (v) of process for the preparation of the new solid-state Form N, the solution is cooled to from about 70° C. to about −10° C., for example, cooled to about room temperature.

In another embodiment of the step (v) process for the preparation of the new solid-state Form N, the crystallization is induced over a time period of from about 15 minutes to about 24 hours. This may be performed with or without stirring the mixture.

In one embodiment of step (vii) of the process for the preparation of the new solid-state Form N, the isolated crystals are dried at a pressure of from about atmospheric pressure to about 5 mbar and at a temperature of from about room temperature to about 100° C. for a time period of from about 1 hour to about 24 hours.

It has been found that by use of the process of the present invention no transformation of the new solid-state Form N takes place and that the Form N product has solid-state purity of greater than about 95.0%, greater than about 99.0%, greater than about 99.9%, or is solid-state pure.

It has also been found that by the use of the process of the present invention no decomposition of the new solid-state Form N takes place and that the Form N product has a chemical purity of greater than about 98.0%, greater than about 99.0%, greater than about 99.5%, or greater than about 99.9%.

It has also been found that the new solid-state Form N is stable under normal storage conditions (typically, but not limited to, temperatures of about 20° C. to about 30° C., and relative humidity of about 30% to about 60%), and does not convert into other known solid-state forms of pantoprazole sodium under crushing or compressing.

The new solid-state Form N solvent free pantoprazole hexacoordinated octahedral sodium aqua complex of the present invention can be converted to the solid-state monohydrate and sesquihydrate forms of pantoprazole sodium, i.e., it may be used as a raw material for the preparation of the solid-state monohydrate and sesquihydrate forms of pantoprazole sodium.

The new solid-state Form N can be also converted, by the use of the processes of the present invention, to the new solid-state solvate forms of pantoprazole hexacoordinated octahedral sodium aqua complexes and to the new solid-state solvate forms of pantoprazole pentacoordinated square pyramidal sodium aqua complexes, described herein

The new solid-state Form N, prepared according to the process of the present invention, can be converted into other pharmaceutically acceptable salts of pantoprazole by means of conventional processes, for example, it may be used as a raw material for preparation of the magnesium salt of pantoprazole.

Solid-State Form A1

Another object of the present disclosure is to provide a new solid-state acetone solvate form of a hexacoordinated octahedral sodium aqua complex of pantoprazole, solid-state Form A1.

The new solid-state acetone solvate Form A1, prepared according to the process of the present invention, has the form of a flowable crystalline powder having the property of flowability, i.e. it is obtained in a “free-flow” form which is not statically chargeable.

Single crystals of the new solid-state acetone solvate Form A1 were prepared according to the process set forth herein, and single crystal x-ray diffraction data collected using a Bruker Nonius FR591/KappaCCD diffractometer using CuKα radiation. Basic crystallographic data for the new solid-state Form A1 are represented in Table 2.

TABLE 2
Basic crystallographic data for the new solid-state
acetone solvent Form A1 hexacoordinated
octahedral sodium aqua complex of pantoprazole.
Form A1

| | |
|---|---|
| Empirical formula | [Na₂(C₁₆H₁₄F₂N₃O₄S)₂(OH₂)₄]. |
| | (C₃H₆O)₂ |
| Formula weight | 998.92 |
| Temperature | 100 (2) K |
| Crystal size | 0.01 × 0.20 × 0.50 mm |
| Crystal system, space | Monoclinic, P 2₁ |
| group | |
| Unit cell dimensions | a = 13.58(2) Å |
| | b = 10.63(1) Å |
| | c = 15.72(2) Å |
| | β = 90.5(3)° |
| | α = γ = 90° |
| Volume | 2269.9(2) Å³ |
| Z | 2 |
| Calculated density | 1.46 gcm⁻³ |

The new solid-state acetone solvate Form A1 has a characteristic x-ray powder pattern, obtained by x-ray diffraction on a powder sample of Form A1. X-ray powder patterns were collected using a Philips X'PertPRO powder diffractometer using CuKα radiation.

The new solid-state acetone solvate Form A1 hexacoordinated octahedral sodium aqua complex of pantoprazole has characteristic x-ray powder diffraction peaks designated by “2Θ” and expressed in degrees as follows: 5.6±0.2°, 11.9±0.2°, 12.9±0.2°, 13.8±0.2°, 15.4±0.2°, 16.4±0.2° and 26.1±0.2°.

The new solid-state acetone solvate Form A1 can be obtained by crystallization from a solution of pantoprazole sodium salt and acetone. A process for preparation of the new solid-state acetone solvate Form A1 hexacoordinated octahedral sodium aqua complex of pantoprazole comprises:
(i) suspending pantoprazole sodium salt in acetone;
(ii) dissolving the pantoprazole sodium salt in acetone;
(iii) optionally filtering the solution of pantoprazole sodium salt and acetone;
(iv) crystallizing the new solid-state acetone solvate Form A1 hexacoordinated octahedral sodium aqua complex of pantoprazole;
(v) isolating the crystals thus obtained; and
(vi) drying the crystals.

In one embodiment of step (ii) of the process for the preparation of the new solid-state acetone solvate Form A1, the suspension of pantoprazole sodium salt and acetone is heated to a temperature of from about 30° C. to about reflux for a time sufficient to obtain clear solution.

In one embodiment of step (iv) of the process for the preparation of the new solid-state acetone solvate Form A1, the solution is cooled to from about 70° C. to about −10° C., for example, cooled to about room temperature.

In another embodiment of step (iv) of the process for the preparation of the new solid-state acetone solvate Form A1, crystallization is induced over a time period of from about 15 minutes to about 24 hours. In one embodiment, this is performed without stirring the mixture.

In one embodiment of step (vi) of the process for the preparation of the new solid-state acetone solvate Form A1, the isolated crystals are dried at about atmospheric pressure and at about room temperature for a time period of from about 1 hour to about 24 hours, for example, for a time period of about 12 hours.

It has been found that by the use of the process of the present invention, no decomposition of the new solid-state acetone solvate Form A1, takes place and that the Form A1 product has a chemical purity of greater than about 98.0%, greater than about 99.0%, greater than about 99.5%, or greater than about 99.9%.

It has also been found that the new solid-state Form A1 is stable under normal storage conditions (typically, but not limited to, temperatures of about 20° C. to about 30° C., and relative humidity of about 30% to about 60%), and does not convert into other known solid-state forms of pantoprazole sodium under crushing or compressing.

The new solid-state acetone solvate Form A1 can be converted to the solid-state monohydrate and sesquihydrate forms of pantoprazole sodium salt, i.e. it may be used as a raw material for the preparation of the solid-state monohydrate and sesquihydrate forms of pantoprazole sodium salt.

The new solid-state acetone solvate Form A1 can be also converted by the use of the processes of the present invention to the new solid-state solvate forms of pantoprazole hexacoordinated octahedral sodium aqua complexes and to the new solid-state solvate forms of pantoprazole pentacoordinated square pyramidal sodium aqua complexes described herein.

The new solid-state acetone solvate Form A1 can be converted into other pharmaceutically acceptable salts of pantoprazole by means of conventional processes, for example, it may be used as a raw material for the preparation of magnesium salt of pantoprazole.

Solid-State Form A2

Another object of this disclosure is to provide a new solid-state acetone solvate pentacoordinated square pyramidal sodium aqua complex of pantoprazole, solid-state Form A2.

The new solid-state acetone solvate Form A2, prepared according to the process of the present invention has the form of a flowable crystalline powder having the property of flowability, i.e. it is obtained in a “free-flow” form which is not statically chargeable.

Single crystals of the new solid-state acetone solvate Form A2 were prepared according to the process of the present invention, and single crystal x-ray diffraction data collected using a Bruker Nonius FR591/KappaCCD diffractometer using CuKα radiation. Basic crystallographic data for the new solid-state acetone solvate Form A2 hexacoordinated octahedral sodium aqua complex of pantoprazole are represented in Table 3.

TABLE 3
Basic crystallographic data for the new solid-state
acetone solvate Form A2 pentacoordinated
square pyramidal sodium aqua complex of pantoprazole.
Form A2

| | |
|---|---|
| Empirical formula | [Na₂(C₁₆H₁₄F₂N₃O₄S)(OH₂)].(C₃H₆O) |
| Formula weight | 481.45 |
| Temperature | 100 (2) K |
| Crystal size | 0.10 × 0.40 × 0.60 mm |
| Crystal system, space | Monoclinic, P 2₁/a |
| group | |
| Unit cell dimensions | a = 13.18(1) Å |
| | b = 10.27(1) Å |
| | c = 17.28(2) Å |
| | β = 109.1(1)° |
| | α = γ = 90° |
| Volume | 2209.4(1) Å³ |
| Z | 4 |
| Calculated density | 1.45 gcm⁻³ |

The new solid-state acetone solvate Form A2 has a characteristic x-ray powder pattern, obtained by x-ray diffraction on a powder sample of Form A2. X-ray powder patterns were collected using a Philips X'PertPRO powder diffractometer using CuKα radiation.

The new solid-state acetone solvate Form A2 has characteristic x-ray powder diffraction peaks, designated by “2Θ” and expressed in degrees, as follows: 5.4±0.2°, 11.3±, 13.8±0.2°, 17.1±0.2°, 23.3±0.2°and 27.1±0.2°.

The new solid-state acetone solvate Form A2 of the present invention can be obtained by crystallization from solutions of pantoprazole sodium salt and acetone. A process for preparation of the new solid-state acetone solvate Form A2 pentacoordinated square pyramidal sodium aqua complex of pantoprazole comprises:
(i) suspending pantoprazole sodium salt in acetone;
(ii) dissolving the pantoprazole sodium salt in acetone;
(iii) optionally filtering the solution of pantoprazole sodium salt and acetone;
(iv) crystallizing the new solid-state acetone solvate Form A2 pentacoordinated square pyramidal sodium aqua complex of pantoprazole;
(v) isolating the crystals thus obtained; and
(vi) drying the crystals.

In one embodiment of stage (ii) of the process for the preparation of the new solid-state acetone solvate Form A2, the suspension of pantoprazole sodium salt and acetone is heated to a temperature of from about 30° C. to about reflux for a time sufficient to obtain a clear solution.

In one embodiment of stage (iv) of the process for the preparation of the new solid-state acetone solvate Form A2, the solution is cooled to from about 70° C. to about −10° C., for example, cooled to about room temperature.

In another embodiment of stage (iv) of the process for the preparation of the new solid-state acetone solvate Form A2, the crystallization is induced over time period of about 15 minutes to about 24 hours. In one embodiment, this is performed without stirring the mixture.

In one embodiment of stage (vi) of the process for the preparation of the new solid-state acetone solvate Form A2, the isolated crystals are dried at about atmospheric pressure and about room temperature for a time period of from about 1 hour to about 24 hours, for example, for a time period of about 12 hours.

It has been found that by the use of the process of the present invention no decomposition of the new solid-state acetone solvate Form A2 pentacoordinated square pyramidal sodium aqua complex of pantoprazole takes place and that it has a chemical purity of greater than about 98.0%, greater than about 99.0%, greater than about 99.5%, or greater than about 99.9%.

It has also been found that the new solid-state Form A2 is stable under normal storage conditions (typically, but not limited to, temperatures of about 20° C. to about 30° C., and relative humidity of about 30% to about 60%), and does not convert into other known solid-state forms of pantoprazole sodium under crushing or compressing.

The new solid-state acetone solvate Form A2 pentacoordinated square pyramidal sodium aqua complex of pantoprazole can be converted to the solid-state monohydrate and sesquihydrate forms of pantoprazole sodium salt, i.e. it may be used as a raw material for the preparation of the solid-state forms monohydrate and sesquihydrate forms of pantoprazole sodium salt.

The new solid-state acetone solvate Form A2 pentacoordinated square pyramidal sodium aqua complex of pantoprazole can be also converted by the use of the processes of the present invention to the new solid-state solvate forms of pantoprazole hexacoordinated octahedral sodium aqua complexes and to the new solid-state solvate forms of pantoprazole pentacoordinated square pyramidal sodium aqua complexes described herein.

The new solid-state acetone solvate Form A2 pentacoordinated square pyramidal sodium aqua complex of pantoprazole can be converted into other pharmaceutically acceptable salts of pantoprazole by means of conventional processes, for example, it may be used as a raw material for the preparation of the magnesium salt of pantoprazole.

Solid-State Form A3

Another object of this disclosure is to provide a new solid-state acetone solvate pantoprazole sodium aqua complex, solid-state Form A3.

The new solid-state acetone solvate Form A3, prepared according to the process of the present invention, has the form of a flowable crystalline powder having the property of flowability, i.e. it is obtained in a “free-flow” form which is not statically chargeable.

The new solid-state acetone solvate Form A3 has a characteristic x-ray powder pattern obtained by x-ray diffraction on a powder sample of the new solid-state acetone solvate Form A3. X-ray powder patterns were collected using a Philips X'PertPRO powder diffractometer using CuKα radiation.

The new solid-state acetone solvate Form A3 has characteristic x-ray powder diffraction peaks designated by “2Θ” and expressed in degrees as follows: 5.4±0.2°; 11.2±0.2°; 16.9±0.2°; 17.6±0.2°; 19.5±0.2°and 26.2±0.2°.

The new solid-state acetone solvate Form A3 of the present invention can be obtained by crystallization from solution of pantoprazole sodium salt and acetone. A process for the preparation of the new solid-state acetone Form A3 sodium aqua complex pantoprazole comprises:
(i) suspending pantoprazole sodium salt in acetone;
(ii) dissolving the pantoprazole sodium salt in acetone;
(iii) optionally filtering the solution of pantoprazole sodium salt and acetone;
(iv) crystallizing the new solid-state acetone Form A3 sodium aqua complex pantoprazole;
(v) isolating the crystals thus obtained; and
(vi) drying the crystals.

In one embodiment of stage (ii) of the process for the preparation of the new solid-state acetone solvate Form A3, the suspension of pantoprazole sodium salt and acetone is heated to a temperature of from about 30° C. to about reflux for a time sufficient to obtain a clear solution.

In one embodiment of stage (iv) of the process for the preparation of the new solid-state acetone solvate Form A3, the solution is cooled to from about 70° C. to about −10° C., for example, cooled to room temperature.

In another embodiment of stage (iv) of the process for the preparation of the new solid-state acetone solvate Form A3, the crystallization is induced over a time period of from about 15 minutes to about 10 hours, for example, over a time period of about 5 hours. In one embodiment, this is performed while stirring the mixture.

In one embodiment of stage (vi) of the process for the preparation of the new solid-state acetone solvate Form A3, the isolated crystals are dried at about atmospheric pressure and about room temperature for a time period of from about 1 hour to about 24 hours, for example, for a time period of about 12 hours.

It has been found that by the use of the process of the present invention no decomposition of the new solid-state acetone solvate Form A3 takes place, and that the Form A3 product has a chemical purity of greater than about 98.0%, greater than about 99.0%, greater than about 99.5 or greater than about 99.9%.

It has also been found that the new solid-state Form A3 is stable under normal storage conditions (typically, but not limited to, temperatures of about 20° C. to about 30° C., and relative humidity of about 30% to about 60%), and does not convert into other known solid-state forms of pantoprazole sodium under crushing or compressing.

The new solid-state acetone solvate Form A3 can be converted to the solid-state monohydrate and sesquihydrate forms of pantoprazole sodium salt, i.e. it may be used as a raw material for the preparation of the solid-state monohydrate and sesquihydrate forms of pantoprazole sodium salt.

The new solid-state acetone solvate Form A3 can be also converted, by the use of the processes of the present invention, to the new solid-state solvate forms of pantoprazole hexacoordinated octahedral sodium aqua complexes and to the new solid-state solvate forms of pantoprazole pentacoordinated square pyramidal sodium aqua complexes described herein.

The new solid-state acetone solvate Form A3 can be converted into other pharmaceutically acceptable salts of pantoprazole by means of conventional processes, for example, it may be used as a raw material for the preparation of the magnesium salt of pantoprazole.

Solid-State Form A4

Another object of this invention is to provide a new solid-state acetone solvate sodium aqua complex of pantoprazole, solid-state Form A4.

The new solid-state acetone solvate Form A4, prepared according to the process of the present invention, has the form of a flowable crystalline powder having the property of flowability, i.e. it is obtained in a “free-flow” form which is not statically chargeable.

The new solid-state acetone solvate Form A4 has characteristic x-ray powder pattern obtained by x-ray diffraction on a powder sample of Form A4. X-ray powder patterns were collected using a Philips X'PertPRO powder diffractometer using CuKα radiation.

The new solid-state acetone solvate Form A4 has characteristic x-ray powder diffraction peaks designated by “2Θ” and expressed in degrees as follows: 5.6±0.2°, 15.4±0.2°, 16.8±0.2°; 17.3±0.2°; 19.6±0.2°; 20.9±0.2°; 24.5±0.2°; 30.1±0.2° and 30.6±0.2°.

The new solid-state acetone Form A4 can be obtained by crystallization from solutions of pantoprazole sodium salt, acetone, and water. A process for the preparation of new solid-state acetone Form A4 sodium aqua complex of pantoprazole comprises:
(i) suspending pantoprazole sodium salt in acetone;
(ii) dissolving the pantoprazole sodium salt in acetone;
(iii) optionally filtering the solution of pantoprazole sodium salt and acetone;
(iv) adding water;
(v) crystallizing the new solid-state acetone solvate Form A4 sodium aqua complex of pantoprazole;
(vi) isolating the crystals thus obtained; and
(vii) drying the crystals.

According to stage (ii) of the process for the preparation of the new solid-state acetone solvate Form A4, the suspension of pantoprazole sodium salt and acetone is heated to a temperature of from about 30° C. to about reflux for a time sufficient to obtain clear solution.

In one embodiment of stage (iv) of the process for the preparation of the new solid-state acetone solvate Form A4, water can be added in an amount of about 0.1% to about 5% by volume of acetone, for example, in an amount of about 2.5% by volume of acetone.

In one embodiment of stage (v) of the process for the preparation of the new solid-state acetone solvate Form A4, the solution is cooled to from about 70° C. to about −10° C., for example, cooled to about room temperature.

In another embodiment of stage (v) of the process for the preparation of the new solid-state acetone solvate Form A4, the crystallization is induced over a time period of from about 15 minutes to about 10 hours, for example, over a timer period of about 5 hours. In one embodiment, this is performed while stirring the mixture.

In one embodiment of stage (vii) of the process for the preparation of the new solid-state acetone solvate Form A4, the isolated crystals are dried at about atmospheric pressure and about room temperature for a time period of from about 1 hour to about 24 hours, for example, for a time period of about 12 hours.

It has been found that by the use of the process of the present invention no decomposition of the new solid-state acetone solvate Form A4 takes place and that the Form A4 product has a chemical purity of greater than about 98.0%, greater than about 99.0%, greater than about 99.5%, or greater than about 99.9%.

It has also been found that the new solid-state Form A4 is stable under normal storage conditions (typically, but not limited to, temperatures of about 20° C. to about 30° C., and relative humidity of about 30% to about 60%), and does not convert into other known solid-state forms of pantoprazole sodium under crushing or compressing.

The new solid-state acetone solvate Form A4 can be converted to the solid-state monohydrate and sesquihydrate forms of pantoprazole sodium salt, i.e. it may be used as a raw material for the preparation of the solid-state monohydrate and sesquihydrate forms of pantoprazole sodium salt.

The new solid-state acetone solvate Form A4 can also be converted by the use of the processes of the present invention to the new solid-state solvate forms of pantoprazole hexacoordinated octahedral sodium aqua complexes and to the new solid-state solvate forms of pantoprazole pentacoordinated square pyramidal sodium aqua complexes described herein.

The new solid-state acetone solvate Form A4 can be converted into other pharmaceutically acceptable salts of pantoprazole by means of conventional processes, for example, it may be used as a raw material for the preparation of the magnesium salt of pantoprazole.

Solid-State Form B1

Still another object of this disclosure is to provide a new solid-state methyl acetate solvate hexacoordinated octahedral sodium aqua complex of pantoprazole, solid-state Form B1.

The new solid-state methyl acetate solvate Form B1, prepared according to the processes of the present invention, has the form of a flowable crystalline powder having the property of flowability, i.e. it is obtained in a “free-flow” form which is not statically chargeable.

Single crystals of the new solid-state methyl acetate solvate Form B1 were prepared by the process of the present invention, and single crystal x-ray diffraction data collected using a Bruker Nonius FR591/KappaCCD diffractometer using CuKα radiation. Basic crystallographic data for the new solid-state methyl acetate solvate Form B1 are represented in Table 4.

TABLE 4
Basic crystallographic data for the new solid-state
methyl acetate solvate Form B1 hexacoordinated
octahedral sodium aqua complex of pantoprazole.
Form B1

| | |
|---|---|
| Empirical formula | [Na(C₁₆H₁₄F₂N₃O₄S)(OH₂)].(C₃H₆O₂) |
| Formula weight | 497.45 |
| Temperature | 293 (2) K |
| Crystal size | 0.15 × 0.20 × 0.40 mm |
| Crystal system, space | Monoclinic, P 2₁/a |
| group | |
| Unit cell dimensions | a = 13.31(1) Å |
| | b = 10.47(1) Å |
| | c = 17.68(2) Å |
| | β = 109.9(1)° |
| | α = γ = 90° |
| Volume | 2316.8(1) Å³ |
| Z | 4 |
| Calculated density | 1.43 gcm⁻³ |

The new solid-state methyl acetate solvate Form B1 has a characteristic x-ray powder pattern obtained by x-ray diffraction on a powder sample of the new solid-state methyl acetate solvate Form B1. X-ray powder patterns were collected using a Philips X'PertPRO powder diffractometer using CuKα radiation.

The new solid-state methyl acetate solvate Form B1 has characteristic x-ray powder diffraction peaks, designated by “2Θ” and expressed in degrees as follows: 5.3±0.2°, 9.9±0.2°, 11.1±0.2°, 13.3±0.2°, 15.8±0.2°, 19.8±0.2°, 21.4±0.2°, 26.1±0.2°, 26.5±0.2°, 28.90.2°and 30.5±0.2°.

The new solid-state methyl acetate solvate Form B1 of the present invention can be obtained by crystallization from solutions of pantoprazole sodium salt and methyl acetate. A process for the preparation of the new solid-state methyl acetate solvate Form B1 hexacoordinated octahedral sodium aqua complex of pantoprazole comprises:
(i) suspending pantoprazole sodium salt in methyl acetate;
(ii) dissolving the pantoprazole sodium salt in methyl acetate;
(iii) optionally filtering the solution of pantoprazole sodium salt and methyl acetate;
(iv) crystallizing the new solid-state methyl acetate solvate Form B1 hexacoordinated octahedral sodium aqua complex of pantoprazole;
(v) isolating the crystals thus obtained; and
(vi) drying the crystals.

In one embodiment of stage (ii) of the process for the preparation of the new solid-state methyl acetate solvate Form B1, the suspension of pantoprazole sodium salt and methyl acetate is heated to a temperature of from about 30° C. to about reflux for a time sufficient to obtain a clear solution.

In one embodiment of stage (iv) of the process for the preparation of the new solid-state methyl acetate solvate Form B1, the solution is cooled to from about 70° C. to about −10° C., for example, cooled to about room temperature.

In another embodiment of stage (iv) of the process for the preparation of the new solid-state methyl acetate solvate Form B1, the crystallization is induced over a period of time from about 15 minutes to about 24 hours. In one embodiment, this is performed without stirring the mixture.

In one embodiment of stage (vi) of the process for the preparation of the new solid-state methyl acetate solvate Form B1, the isolated crystals are dried at about atmospheric pressure and about room temperature for a time period of from about 1 hour to about 24 hours, for example, for a time period of about 12 hours.

It has been found that by the use of the process of the present invention, no decomposition of the new solid-state methyl acetate solvate Form B1 takes place and that the Form B1 product has a chemical purity of greater than about 98.0%, greater than about 99.0%, greater than about 99.5%, or greater than about 99.9%.

It has also been found that the new solid-state Form B1 is stable under normal storage conditions (typically, but not limited to, temperatures of about 20° C. to about 30° C., and relative humidity of about 30% to about 60%), and does not convert into other known solid-state forms of pantoprazole sodium under crushing or compressing.

The new solid-state methyl acetate solvate Form B1 can be converted to the solid-state monohydrate and sesquihydrate forms of pantoprazole sodium salt, i.e., it may be used as a raw material for the preparation of the solid-state monohydrate and sesquihydrate forms of pantoprazole sodium salt.

The new solid-state methyl acetate solvate Form B1 can be also converted by the use of the processes of the present invention, to the new solid-state solvate forms of pantoprazole hexacoordinated octahedral sodium aqua complexes and to the new solid-state solvate forms of pantoprazole pentacoordinated square pyramidal sodium aqua complexes described herein.

The new solid-state methyl acetate solvate Form B1 can be converted into other pharmaceutically acceptable salts of pantoprazole by means of conventional processes, for example, it may be used as a raw material for the preparation of the magnesium salt of pantoprazole.

Solid-State Form B2

Another object of this disclosure is to provide a new solid-state methyl acetate solvate sodium aqua complex of pantoprazole, solid-state Form B2.

The new solid-state methyl acetate solvate Form B2, prepared according to the processes of the present invention, has the form of a flowable crystalline powder having the property of flowability, i.e. it is obtained in a “free-flow” form which is not statically chargeable.

The new solid-state methyl acetate solvate Form B2 has a characteristic x-ray powder pattern obtained by x-ray diffraction on a powder sample of the new solid-state methyl acetate solvate Form B2. X-ray powder patterns were collected using a Philips X'PertPRO powder diffractometer using CuKα radiation.

The new solid-state methyl acetate solvate Form B2 has characteristic x-ray powder diffraction peaks designated by “2Θ” and expressed in degrees as follows: 5.4±0.2°, 11.2±0.2°, 13.3±0.2°, 16.8±0.2°, 20.5±0.2°, 22.4±0.2°and 26.6±0.2°.

The new solid-state methyl acetate solvate Form B2 of the present invention can be obtained by crystallization from solutions of pantoprazole sodium salt and methyl acetate. A process for preparation of the new solid-state methyl acetate solvate Form B2 sodium aqua complex of pantoprazole comprises:
(i) suspending pantoprazole sodium salt in methyl acetate;
(ii) dissolving the pantoprazole sodium salt in methyl acetate;
(iii) optionally filtering the solution of pantoprazole sodium salt and methyl acetate;
(iv) crystallizing the new solid-state methyl acetate solvate Form B2 sodium aqua complex of pantoprazole;
(v) isolating the crystals thus obtained; and
(vi) drying the crystals.

In one embodiment of stage (ii) of the process for the preparation of the new solid-state methyl acetate solvate Form B2, the suspension of pantoprazole sodium salt and methyl acetate is heated to a temperature of from about 30° C. to about reflux for a time sufficient to obtain clear solution.

In one embodiment of stage (iv) of the process for the preparation of the new solid-state methyl acetate solvate Form B2, the solution is cooled to from about 70° C. to about −10° C., for example, cooled to about room temperature.

In one embodiment of stage (iv) of the process for the preparation of the new solid-state methyl acetate solvate Form B2, the crystallization is induced over a time period of from about 15 minutes to about 10 hours, preferably over a time period of about 5 hours.

In another embodiment of stage (vi) of the process for the preparation of the new solid-state methyl acetate solvate Form B2, the isolated crystals, are dried at about atmospheric pressure and about room temperature for a time period of from about 1 hour to about 24 hours, for example, for a timer period of about 12 hours. In one embodiment, this is performed while stirring the mixture.

It has been found that by the use of the process of the present invention no decomposition of the new solid-state methyl acetate solvate Form B2 takes place and that the Form B2 product has a chemical purity of greater than about 98.0%, greater than about 99.0%, greater than about 99.5%, or greater than about 99.9%.

It has also been found that the new solid-state Form B2 is stable under normal storage conditions (typically, but not limited to, temperatures of about 20° C. to about 30° C., and relative humidity of about 30% to about 60%), and does not convert into other known solid-state forms of pantoprazole sodium under crushing or compressing.

The new solid-state methyl acetate solvate Form B2 can be converted to the solid-state monohydrate and sesquihydrate forms of pantoprazole sodium salt, i.e. it may be used as a raw material for the preparation of the solid-state monohydrate and sesquihydrate forms of pantoprazole sodium salt.

The new solid-state methyl acetate solvate Form B2 can also be also converted by the use of the processes of the present invention to the new solid-state solvate forms of pantoprazole hexacoordinated octahedral sodium aqua complexes and to the new solid-state solvate forms of pantoprazole pentacoordinated square pyramidal sodium aqua complexes described herein.

The new solid-state methyl acetate solvate Form B2 can be converted into other pharmaceutically acceptable salts of pantoprazole by means of conventional processes, for example, it may be used as a raw material for the preparation of the magnesium salt of pantoprazole.

Solid-state Form B3

Another object of this disclosure is to provide a new solid-state methyl acetate solvate sodium aqua complex of pantoprazole, solid-state Form B3.

The new solid-state methyl acetate solvate Form B3, prepared according to the process of the present invention, has the form of a flowable crystalline powder having the property of flowability, i.e. it is obtained in a “free-flow” form which is not statically chargeable.

The new solid-state methyl acetate solvate Form B3 of the present invention has a characteristic x-ray powder pattern obtained by x-ray diffraction on a powder sample of the new solid-state methyl acetate solvate Form B3. X-ray powder patterns were collected using a Philips X'PertPRO powder diffractometer using CuKα radiation.

The new solid-state methyl acetate solvate Form B3 has characteristic x-ray powder diffraction peaks designated by “2Θ” and expressed in degrees as follows: 5.5±0.2°, 9.5±0.2°, 11.9±0.2°, 15.3±0.2°, 19.2±0.2°, 23.9±0.2°and 33.0±0.2°.

The new solid-state methyl acetate solvate Form B3 of present invention can be obtained by crystallization from solutions of pantoprazole sodium salt, methyl acetate and water. A process for the new solid-state methyl acetate solvate Form B3 sodium aqua complex of pantoprazole comprises:
(i) suspending pantoprazole sodium salt in methyl acetate;
(ii) dissolving the pantoprazole sodium salt in methyl acetate;
(iii) optionally filtering the solution of pantoprazole sodium salt and methyl acetate;
(iv) adding water;
(v) crystallizing the new solid-state methyl acetate Form B3 sodium aqua complex of pantoprazole;
(vi) isolating the crystals thus obtained; and
(vii) drying the crystals.

In one embodiment of stage (ii) of the process for the preparation of the new solid-state methyl acetate solvate Form B3, the suspension of pantoprazole sodium salt and methyl acetate is heated to a temperature of from about 30° C. to about reflux for a time sufficient to obtain clear solution.

In one embodiment of stage (iv) of the process for the preparation of the new solid-state methyl acetate solvate Form B3, water can be added in an amount of about 0.1% to about 5% by volume of methyl acetate, for example, in an amount of about 2.5% by volume of methyl acetate.

In one embodiment of stage (v) of the process for the preparation of the new solid-state methyl acetate solvate Form B3, the solution is cooled to from about 70° C. to about −10° C., for example, cooled to room temperature.

In another embodiment of stage (v) of the process for the preparation of the new solid-state methyl acetate solvate Form B3, the crystallization is induced over a time period of from about 15 minutes to about 10 hours, for example, over a time period of about 5 hours. In one embodiment, this is performed while stirring the mixture.

In one embodiment of stage (vii) of the process for the preparation of the new solid-state methyl acetate solvate Form B3, the isolated crystals are dried at about atmospheric pressure and about room temperature for a time period of from about 1 hour to about 24 hours, for example for a time period of about 12 hours.

It has been found that by the use of the process of the present invention no decomposition of the new solid-state methyl acetate solvate Form B3 takes place and that the Form B3 product has a chemical purity of greater than about 98.0%, greater than about 99.0%, greater than about 99.5%, or greater than about 99.9%.

It has also been found that the new solid-state Form B3 is stable under normal storage conditions (typically, but not limited to, temperatures of about 20° C. to about 30° C., and relative humidity of about 30% to about 60%), and does not convert into other known solid-state forms of pantoprazole sodium under crushing or compressing.

The new solid-state methyl acetate solvate Form B3 can be converted to the solid-state monohydrate and sesquihydrate forms of pantoprazole sodium salt, i.e. it may be used as a raw material for the preparation of the solid-state monohydrate and sesquihydrate forms of pantoprazole sodium salt.

The new solid-state methyl acetate solvate Form B3 can also be converted by the use of the processes of the present invention to the new solid-state solvate forms of pantoprazole hexacoordinated octahedral sodium aqua complexes and to the new solid-state solvate forms of pantoprazole pentacoordinated square pyramidal sodium aqua complexes described herein.

The new solid-state methyl acetate solvate Form B3 can be converted into other pharmaceutically acceptable salts of pantoprazole by means of conventional processes, for example, it may be used as a raw material for the preparation of the magnesium salt of pantoprazole.

Solid-State Form C1

Still another object of this disclosure is to provide a new solid-state methyl ethyl ketone solvate hexacoordinated octahedral sodium aqua complex of pantoprazole, solid-state Form C1.

The new solid-state methyl ethyl ketone solvate Form C1, prepared according to the process of the present invention, has the form of a flowable crystalline powder having the property of flowability, i.e. it is obtained in a “free-flow” form which is not statically chargeable.

Single crystals of the new solid-state methyl ethyl ketone solvate Form C1 were prepared by the process of the present invention, and single crystal x-ray diffraction data collected using a Bruker Nonius FR591/KappaCCD diffractometer using CuKα radiation. Basic crystallographic data for the new solid-state methyl acetate solvate Form C1 are represented in Table 5.

TABLE 5
Basic crystallographic data for the new solid-state methyl
ethyl ketone solvate Form C1 hexacoordinated
octahedral sodium aqua complex of pantoprazole.
Form C1

| | |
|---|---|
| Empirical formula | [Na(C₁₆H₁₄F₂N₃O₄S)(OH₂)₂] × CH₃CH₂COCH₃ |
| Formula weight | 513.49 |
| Temperature | 293 (2) K |
| Crystal size | 0.05 × 0.1 × 0.20 mm |
| Crystal system, space | Monoclinic, P 2₁/a |
| group | |
| Unit cell dimensions | a = 13.51(1) Å |
| | b = 10.66(1) Å |
| | c = 16.16(2) Å |
| | β = 92.3(1)° |
| | α = γ = 90° |
| Volume | 2324.8(10) Å³ |
| Z | 4 |
| Calculated density | 1.47 gcm⁻³ |

The new solid-state methyl ethyl ketone Form C1 has a characteristic x-ray powder pattern obtained by x-ray diffraction on a powder sample of the new solid-state methyl ethyl ketone solvate Form C1. X-ray powder patterns were collected using a Philips X'PertPRO powder diffractometer using CuKα radiation.

The new solid-state methyl ethyl ketone solvate Form C1 has characteristic x-ray powder diffraction peaks designated by “20” and expressed in degrees as follows: 5.5±0.2°, 10.4±0.2°, 10.9±0.2°, 19.2±0.2°, 20.5±0.2°, 21.4±0.2°, 24.6±0.2°, 29.7±0.2°, 33.0±0.2°and 33.9±0.2°.

The new solid-state methyl ethyl ketone solvate Form C1 of the present invention can be obtained by crystallization from solutions of pantoprazole sodium salt and methyl ethyl ketone.

A process for the preparation of the new solid-state methyl ethyl ketone solvate Form C1 hexacoordinated octahedral sodium aqua complex of pantoprazole comprises:
(i) suspending pantoprazole sodium salt in methyl ethyl ketone;
(ii) dissolving the pantoprazole sodium salt in methyl ethyl ketone;
(iii) optionally filtering the solution of pantoprazole sodium salt and methyl ethyl ketone;
(iv) optionally adding water
(v) crystallizing the new solid-state methyl ethyl ketone solvate Form C1 hexacoordinated octahedral sodium aqua complex of pantoprazole;
(vi) isolating the crystals thus obtained; and
(vii) drying the crystals.

In one embodiment of stage (ii) of the process for the preparation of the new solid-state methyl ethyl ketone solvate Form C1, the suspension of pantoprazole sodium salt and methyl ethyl ketone is heated to a temperature of from about 30° C. to about reflux for a time sufficient to obtain a clear solution.

In one embodiment of step (iv) of the process for the preparation of the new solid-state methyl ethyl ketone solvate Form C1, water can be added in an amount of about 0.1% to about 5% by volume of the methyl ethyl ketone, for example, in an amount of about 2.5% by volume of the methyl ethyl ketone.

In one embodiment of stage (v) of the process for the preparation of the new solid-state methyl ethyl ketone solvate Form C1, the solution is cooled to from about 70° C. to about −10° C., for example, cooled to about room temperature.

In another embodiment of stage (v) of the process for the preparation of the new solid-state methyl ethyl ketone solvate Form C1, the crystallization is induced over a period of time of from about 15 minutes to about 24 hours. In one embodiment, this is performed without stirring the mixture.

In one embodiment of stage (vii) of the process for the preparation of the new solid-state methyl ethyl ketone solvate Form C1, the isolated crystals are dried at about atmospheric pressure and about room temperature for a time period of from about 1 hour to about 24 hours, for example, for a time period of about 12 hours.

It has been found that by the use of the process of the present invention, no decomposition of the new solid-state methyl ethyl ketone solvate Form C1, takes place and that the Form C1 product has a chemical purity of greater than about 98.0%, greater than about 99.0%, greater than about 99.5%, or greater than about 99.9%.

It has also been found that the new solid-state Form C1 is stable under normal storage conditions (typically, but not limited to, temperatures of about 20° C. to about 30° C., and relative humidity of about 30% to about 60%), and does not convert into other known solid-state forms of pantoprazole sodium under crushing or compressing.

The new solid-state methyl ethyl ketone solvate Form C1 can be converted to the solid-state monohydrate and sesquihydrate forms of pantoprazole sodium salt, i.e. it may be used as a raw material for the preparation of the solid-state monohydrate and sesquihydrate forms of pantoprazole sodium salt.

The new solid-state methyl ethyl ketone solvate Form C1 can also be converted by the use of the processes of the present invention, to the new solid-state solvate forms of pantoprazole hexacoordinated octahedral sodium aqua complexes and to the new solid-state solvate forms of pantoprazole pentacoordinated square pyramidal sodium aqua complexes describer herein.

The new solid-state methyl ethyl ketone solvate Form C1 can be converted into other pharmaceutically acceptable salts of pantoprazole by means of conventional processes, for example, it may be used as a raw material for the preparation of the magnesium salt of pantoprazole.

Solid-State Form C2

Another object of this disclosure is to provide a new solid-state methyl ethyl ketone solvate sodium aqua complex of pantoprazole, solid-state Form C2.

The new solid-state methyl ethyl ketone solvate Form C2, prepared according to the process of the present invention, has the form of a flowable crystalline powder having the property of flowability, i.e. it is obtained in a “free-flow” form which is not statically chargeable.

The new solid-state methyl ethyl ketone solvate Form C2 of the present invention has a characteristic x-ray powder pattern obtained by x-ray diffraction on a powder sample of the new solid-state methyl ethyl ketone solvate Form C2. X-ray powder patterns were collected using a Philips X'PertPRO powder diffractometer using CuKα radiation.

The new solid-state methyl ethyl ketone solvate Form C2 has characteristic x-ray powder diffraction peaks designated by “2Θ” and expressed in degrees as follows: 5.4±0.2°, 10.7±0.2°, 12.3±0.2°, 15.8±0.2°, 16.7±0.2°, 20.1±0.2° and 22.5±0.2°.

The new solid-state methyl ethyl ketone solvate Form C2 can be obtained by crystallization from solutions of pantoprazole sodium salt and methyl ethyl ketone. A process for the preparation of new solid-state methyl ethyl ketone solvate Form C2 sodium aqua complex of pantoprazole comprises:
(i) suspending pantoprazole sodium salt in methyl ethyl ketone;
(ii) dissolving the pantoprazole sodium salt in methyl ethyl ketone;
(iii) optionally filtering the solution of pantoprazole sodium salt and methyl ethyl ketone;
(iv) crystallizing the new solid-state methyl ethyl ketone solvate Form C2 sodium aqua complex of pantoprazole;
(v) isolating the crystals thus obtained; and
(vi) drying the crystals.

In one embodiment of stage (ii) of the process for the preparation of the new solid-state methyl ethyl ketone solvate Form C2, the suspension of pantoprazole sodium salt and methyl ethyl ketone is heated to a temperature of from about 30° C. to about reflux for a time sufficient to obtain clear solution.

In one embodiment of stage (iv) of the process for the preparation of the new solid-state methyl ethyl ketone solvate Form C2, the solution is cooled to from about 70° C. to about −10° C., for example cooled to room temperature.

In another embodiment of stage (iv) of the process for the preparation of the new solid-state methyl ethyl ketone solvate Form C2, the crystallization is induced over a time period of from about 15 minutes to about 10 hours, for example, over a time period of about 5 hours. In one embodiment, this is performed while stirring the mixture.

In one embodiment of stage (vi) of the process for the preparation of the new solid-state methyl ethyl ketone solvate Form C2, the isolated crystals are dried at about atmospheric pressure and about room temperature for a time period of from about 1 hour to about 24 hours, for example, for a time period of about 12 hours.

It has been found that by the use of the process of the present invention no decomposition of the new solid-state methyl ethyl ketone solvate Form C2 takes place and that the Form C2 product has a chemical purity of greater than about 98.0%, greater than about 99.0%, greater than about 99.5%, or greater than about 99.9%.

It has also been found that the new solid-state Form C2 is stable under normal storage conditions (typically, but not limited to, temperatures of about 20° C. to about 30° C., and relative humidity of about 30% to about 60%), and does not convert into other known solid-state forms of pantoprazole sodium under crushing or compressing.

The new solid-state methyl ethyl ketone solvate Form C2 can be converted to the solid-state monohydrate and sesquihydrate forms of pantoprazole sodium salt, i.e. it may be used as a raw material for the preparation of the solid-state monohydrate and sesquihydrate forms of pantoprazole sodium salt.

The new solid-state methyl ethyl ketone solvate Form C2 can be also converted by the use of the processes of the present invention, to the new solid-state solvate forms of pantoprazole hexacoordinated octahedral sodium aqua complexes and to the new solid-state solvate forms of pantoprazole pentacoordinated square pyramidal sodium aqua complexes describer herein.

The new solid-state methyl ethyl ketone solvate Form C2 can be converted into other pharmaceutically acceptable salts of pantoprazole by means of conventional processes, for example, it may be used as a raw material for the preparation of the magnesium salt of pantoprazole.

Solid-State Form D1

Still another object of this disclosure is to provide a new solid-state diethyl ketone solvate hexacoordinated octahedral sodium aqua complex of pantoprazole, solid-state Form Dl.

The new solid-state diethyl ketone solvate Form D1, prepared according to the processes of the present invention, has the form of a flowable crystalline powder having the property of flowability, i.e. it is obtained in a “free-flow” form which is not statically chargeable.

Single crystals of a new solid-state diethyl ketone solvate Form D1 were prepared and single crystal x-ray diffraction data collected using a Bruker Nonius FR591/KappaCCD diffractometer using CuKα radiation.

Basic crystallographic data for the new solid-state diethyl ketone solvate Form D1 are represented in Table 6.

TABLE 6
Basic crystallographic data for the new solid-state diethyl
ketone solvate Form D1 hexacoordinated octahedral
sodium aqua complex of pantoprazole.
Form D1

| | |
|---|---|
| Empirical formula | [Na(C₁₆H₁₄F₂N₃O₄S)(OH₂)] × (CH₃CH₂)₂CO |
| Formula weight | 527.51 |
| Temperature | 100 (2) K |
| Crystal size | 0.1 × 0.2 × 0.40 mm |
| Crystal system, space | Monoclinic, P 2₁/a |
| group | |
| Unit cell dimensions | a = 13.42(1) Å |
| | b = 10.85(1) Å |
| | c = 17.36(2) Å |
| | β = 102.5(1)° |
| | α = γ = 90° |
| Volume | 2469.0(1) Å³ |
| Z | 4 |
| Calculated density | 1.42 gcm⁻³ |

The new solid-state diethyl ketone solvate Form D1 has a characteristic x-ray powder pattern obtained by x-ray diffraction on a powder sample of the new solid-state diethyl ketone solvate Form D1. X-ray powder patterns were collected using a Philips X'PertPRO powder diffractometer using CuKα radiation.

The new solid-state diethyl ketone solvate Form D1 has characteristic x-ray powder diffraction peaks designated by “2Θ” and expressed in degrees as follows: 5.2±0.2°, 10.4±0.2°, 12.3±0.2°, 13.1±0.2°, 15.1±0.2°, 15.8±0.2°, and 25.0±0.2°.

The new solid-state diethyl ketone solvate Form D1 of the present invention can be obtained by crystallization from solutions of pantoprazole sodium salt and diethyl ketone. A process for the preparation of the new solid-state diethyl ketone solvate Form D1 hexacoordinated octahedral sodium aqua complex of pantoprazole comprises:
(i) suspending pantoprazole sodium salt in diethyl ketone;
(ii) dissolving the pantoprazole sodium salt in diethyl ketone;
(iii) filtering the solution of pantoprazole sodium salt and diethyl ketone;
(iv) crystallizing the new solid-state diethyl ketone solvate Form D1 hexacoordinated octahedral sodium aqua complex of pantoprazole;
(v) isolating the crystals thus obtained; and
(vi) drying the crystals.

In one embodiment of stage (ii) of the process for the preparation of the new solid-state diethyl ketone solvate Form D1, the suspension of pantoprazole sodium salt and diethyl ketone is heated to a temperature of from about 30° C. to about reflux for a time sufficient to obtain clear solution.

In one embodiment of stage (iv) the process for the preparation of the new solid-state diethyl ketone solvate Form D1, the solution is cooled to from about 70° C. to about −10° C., for example, cooled to room temperature.

In another embodiment of stage (iv) of the process for the preparation of the new solid-state diethyl ketone solvate Form D1, the crystallization is induced stirring over a time period of from about 15 minutes to about 24 hours. This may be performed with or without stirring.

In one embodiment of stage (vi) of the process for the preparation of the new solid-state diethyl ketone solvate Form D1, the isolated crystals are dried at about atmospheric pressure and about room temperature for a time period of from about 1 hour to about 24 hours, for example, for a timer period of about 12 hours.

It has been found that by the use of the process of the present invention no decomposition of the new solid-state diethyl ketone solvate Form D1 takes place and that the Form D1 product has a chemical purity of greater than about 98.0%, greater than about 99.0%, greater than about 99.5%, or greater than about 99.9%.

It has also been found that the new solid-state Form D1 is stable under normal storage conditions (typically, but not limited to, temperatures of about 20° C. to about 30° C., and relative humidity of about 30% to about 60%), and does not convert into other known solid-state forms of pantoprazole sodium under crushing or compressing.

The new solid-state diethyl ketone solvate Form D1 can be converted to the solid-state monohydrate and sesquihydrate forms of pantoprazole sodium salt, i.e. it may be used as a raw material for the preparation of the solid-state monohydrate and sesquihydrate forms of pantoprazole sodium salt.

The new solid-state diethyl ketone solvate Form D1 can also be converted by the use of the processes of the present invention to the new solid-state solvate forms of pantoprazole hexacoordinated octahedral sodium aqua complexes and to the new solid-state solvate forms of pantoprazole pentacoordinated square pyramidal sodium aqua complexes describer herein.

The new solid-state diethyl ketone solvate Form D1 can be converted into other pharmaceutically acceptable salts of pantoprazole by means of conventional processes, for example, it may be used as a raw material for the preparation of the magnesium salt of pantoprazole.

Solid-State Form E1

Still another object of this disclosure is to provide a desolvated sodium aqua complex of pantoprazole, solid-state Form E1.

The desolvated Form E1, prepared according to the processes of the present invention, has the form of a flowable crystalline powder having the property of flowability, i.e. it is obtained in a “free-flow” form which is not statically chargeable.

The desolvated Form E1 has a characteristic x-ray powder pattern obtained by x-ray diffraction on a powder sample of the desolvated Form E1. X-ray powder patterns were collected using a Philips X'PertPRO powder diffractometer using CuKα radiation.

The desolvated Form E1 has characteristic x-ray powder diffraction peaks designated by “20)” and expressed in degrees as follows: 5.4±0.2°, 11.6±0.2, 12.4±0.2°, 13.6±0.2, 16.0±0.2°, 23.3±0.2° and 28.7±0.2°.

The desolvated Form E1 of the present invention can be obtained by drying solvates of pantoprazole sodium aqua complexes, including, but not limited to, the solvates described herein.

A process for the preparation of the desolvated Form E1 comprises drying solvates of pantoprazole sodium aqua complexes at temperatures of from about 20° C. to about 120° C., for example, at about 60° C., and at pressures of from about 1 mbar to about 10 mbar, for example, at about 5 mbar for a time period of from about 1 hour to about 6 hours, for example, for about 3 hours.

The obtained crystals of Form E1 have characteristic x-ray powder diffraction peaks, (2Θ) expressed in degrees, at: 5.4±0.2°, 11.6±0.2°, 12.4±0.2°, 13.6±0.2°, 16.0±0.2°, 23.3±0.2° and 28.7±0.2°.

It has been found that by use of the process of the present invention no transformation of the desolvated Form E1 takes place and the Form E1 product that has a solid-state purity of greater than about 95.0%, greater than about 95.0%, greater than about 99.9%, or that it is solid-state pure.

It has also been found that by the use of the process of the present invention no decomposition of the desolvated Form E1 takes place and that the Form E1 product has a chemical purity of greater than about 98.0%, greater than about 99.0%, greater than about 99.5%, or greater than about 99.9%.

It has also been found that the new solid-state Form E1 is stable under normal storage conditions (typically, but not limited to, temperatures of about 20° C. to about 30° C., and relative humidity of about 30% to about 60%), and does not convert into other known solid-state forms of pantoprazole sodium under crushing or compressing.

The desolvated Form E1 can be converted to the solid-state monohydrate and sesquihydrate forms of pantoprazole sodium salt, i.e. it may be used as a raw material for the preparation of the solid-state monohydrate and sesquihydrate forms of pantoprazole sodium salt.

The desolvated Form E1 can be also converted by the use of the processes of the present invention to the new solid-state solvate forms of pantoprazole hexacoordinated octahedral sodium aqua complexes and to the new solid-state solvate forms of pantoprazole pentacoordinated square pyramidal sodium aqua complexes described herein.

The desolvated Form E1 can be converted into other pharmaceutically acceptable salts of pantoprazole by means of conventional processes, for example, it may be used as a raw material for the preparation of the magnesium salt of pantoprazole.

Compositions of the New Solid-State Forms of Pantoprazole

The new solid-state Forms N, A1, A2, A3, A4, B1, B2, B3, C1, C2, D1, and E1 of sodium aqua complexes of pantoprazole of the present invention can be utilized in the preparation of rapid, controlled and sustained release pharmaceutical compositions, suitable for oral, rectal, parenteral, transdermal, buccal, nasal, sublingual, subcutaneous or intravenous administration. For example, the compostitions may include one or more of solid-state Form N and solid-state Form E1.

The compositions may be administered orally, in the form of rapid or controlled release tablets, microparticles, mini tablets, capsules, sachets, and oral solutions or suspensions, or powders for the preparation thereof. In addition to the new solid-state forms of pantoprazole of the present invention as the active substance, oral preparations may optionally include various standard pharmaceutical carriers and excipients, such as binders, fillers, buffers, lubricants, glidants, dyes, disintegrants, odorants, sweeteners, surfactants, mold release agents, antiadhesive agents and coatings. Some excipients may have multiple roles in the compositions, e.g., act as both binders and disintegrants.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, DL-leucine and colloidal silicon dioxide

Examples of suitable pharmaceutically acceptable odorants for the oral compositions include, but are not limited to, synthetic aromas and natural aromatic oils such as extracts of oils, flowers, fruits and combinations thereof. Examples are vanilla and fruit aromas, including banana, apple, sour cherry, peach and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulfate and polysorbates.

Compositions of the solid-state forms of pantoprazole of the present invention can also be administered intravenously or intraperitoneally, by infusion or injection. Dispersions can also be prepared in a liquid carrier or intermediate, such as glycerin, liquid polyethylene glycols, triacetin oils, and mixtures thereof. To improve storage stability, such preparations may also contain a preservative to prevent the growth of microorganisms.

Pharmaceutical compositions suitable for injection or infusion may be in the form of a sterile aqueous solution, a dispersion or a sterile powder that contains the active ingredient, adjusted, if necessary, for preparation of such a sterile solution or dispersion suitable for infusion or injection. This may optionally be encapsulated into liposomes. In all cases, the final preparation must be sterile, liquid, and stable under production and storage conditions.

The liquid carrier or intermediate can be a solvent or liquid dispersive medium that contains, for example, water, ethanol, a polyol (e.g. glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants. Prevention of the action of micro-organisms can be achieved by the addition of various antibacterial and antifungal agents, e.g. paraben, chlorobutanol, or sorbic acid. In many cases isotonic substances are recommended, e.g. sugars, buffers and sodium chloride to assure osmotic pressure similar to those of body fluids, particularly blood. Prolonged absorption of such injectable mixtures can be achieved by introduction of absorption-delaying agents, such as aluminium monostearate or gelatin.

Sterile injectable solutions can be prepared by mixing the solid-state Forms of pantoprazole with an appropriate solvent and one or more of the aforementioned excipients, followed by sterile filtering. In the case of sterile powders suitable for use in the preparation of sterile injectable solutions, preferable preparation methods include drying in vacuum and lyophilization, which provide powdery mixtures of the isostructural pseudopolymorphs and desired excipients for subsequent preparation of sterile solutions.

The solid-state forms of pantoprazole of the present invention may also be used for the preparation of locally acting, topical compositions. Such compositions may also contain other pharmaceutically acceptable excipients, such as polymers, oils, liquid carriers, surfactants, buffers, preservatives, stabilizers, antioxidants, moisturizers, emollients, colorants and odorants. Examples of pharmaceutically acceptable polymers suitable for such topical compositions include, but are not limited to, acrylic polymers; cellulose derivatives, such as carboxymethylcellulose sodium, methylcellulose or hydroxypropylcellulose; natural polymers, such as alginates, tragacanth, pectin, xanthan and cytosan.

Examples of suitable pharmaceutically acceptable oils which are so useful include but are not limited to, mineral oils, silicone oils, fatty acids, alcohols, and glycols.

Examples of suitable pharmaceutically acceptable liquid carriers include, but are not limited to, water, alcohols or glycols such as ethanol, isopropanol, propylene glycol, hexylene glycol, glycerol and polyethylene glycol, or mixtures thereof in which the pseudopolymorph is dissolved or dispersed, optionally with the addition of non-toxic anionic, cationic or non-ionic surfactants, and inorganic or organic buffers.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetraacetic acid (EDTA), thiourea, tocopherol and butyl hydroxyanisole.

Suitable examples of pharmaceutically acceptable moisturizers include, but are not limited to, glycerine, sorbitol, urea and polyethylene glycol.

Suitable examples of pharmaceutically acceptable emollients include, but are not limited to, mineral oils, isopropyl myristate, and isopropyl palmitate.

The use of dyes and odorants in topical compositions of the present invention depends on many factors of which the most important is organoleptic acceptability to the population that will be using the pharmaceutical compositions.

The therapeutically acceptable quantity of the solid-state forms of pantoprazole of the present invention administered varies, dependent on the selected compound, the mode of administration, treatment conditions, age and status of the patient or animal species, and is subject to the final decision of the physician, clinician or veterinary doctor monitoring the course of treatment. For example, the solid-state forms of pantoprazole may be formulated in a dosage form that contains from about 5 to about 300 mg of the active substance per unit dose.

The present invention also relates to methods for inhibiting gastric acid secretion, protecting the stomach and intestines, and treating gastric ulcers in a patient in need of such treatment by administering to the patient a therapeutically effective amount of one or more of the new solid-state sodium aqua complexes of pantoprazole Forms N, A1, A2, A3, A4, B1, B2, B3, C1, C2, D1, or E1 or a pharmaceutical composition containing a therapeutically effective amount of one or more of the new solid-state sodium aqua complexes of pantoprazole Forms N, A1, A2, A3, A4, B1, B2, B3, C1, C2, D1, and E1. For example, the methods relate to administering one or more of solid-state Form N and solid-state Form E1.

### EXAMPLES

The present invention is illustrated but in no way limited by the following examples.

### Example 1

Pantoprazole sodium (0.4 g) was dissolved in n-butylacetate (5 ml). After cooling to room temperature, the solution was filtered and 0.2 ml of demineralized water was added. The resulting mixture was left at the same temperature for 24 hours. The crystals obtained were separated by suction and dried to yield 0.29 g of Form N crystals.

Basic crystallographic data for the new solid-state Form N complex are represented in Table 1.

The new solid-state Form N complex has characteristic x-ray powder diffraction peaks designated by “2Θ” and expressed in degrees as follows: 5.3±0.2°, 13.1±0.2°, 16.9±0.2°, 20.5±0.2°, 21.6±0.2°and 25.1±0.2°.

### Example 2

Pantoprazole sodium (5.0 g) was dissolved in n-butylacetate (190 ml) and 2.5 ml of water was added. After cooling to room temperature, the solution was filtered and then stirred for 5 hours at the same temperature. The obtained suspension was filtered, separated, and the separated crystals were washed with n-butylacetate and dried at 60° C. under a vacuum of 5 mbar for 3 hours. Yield: 4.6 g of Form N crystals.

The x-ray powder pattern of the thus obtained sample corresponds to the x-ray powder pattern of the solid-state Form N product obtained in Example 1.

### Example 3

Crude pantoprazole sodium (10.0 g) was dissolved in ethylacetate (400 ml) and 2.0 ml of water was added. After cooling to room temperature, the solution was filtered and then stirred for 5 hours at the same temperature. The obtained suspension was filtered, and the separated crystals were washed with ethylacetate and dried at 80° C. under a vacuum of 5 mbar for 1 hour. Yield: 8.7 g of Form N crystals.

The x-ray powder pattern of the thus obtained sample corresponds to the x-ray powder pattern of the solid-state Form N product obtained in Example 1.

### Example 4

Pantoprazole sodium (0.40 g) was dissolved in acetone (10 ml). After cooling to room temperature, the solution was left at the same temperature for 12 hours. The crystals obtained were separated by suction and dried at room temperature and atmospheric pressure for 12 hours, to yield 0.32 g of Form A1 crystals.

Basic crystallographic data for the new solid-state acetone solvate Form A1 are represented in Table 2.

The new solid-state acetone solvate Form A1 has characteristic x-ray powder diffraction peaks designated by “2Θ” and expressed in degrees as follows: 5.6±0.2°, 11.9±0.2°, 12.9±0.2°, 13.8±0.2°, 15.4±0.2°, 16.4±0.2° and 26.1±0.2°.

### Example 5

Crude pantoprazole sodium (0.40 g) was dissolved in acetone (7.5 ml). After cooling to room temperature, the solution was left at the same temperature for 24 hours. The crystals obtained were separated by suction and dried at room temperature and atmospheric pressure for 6 hours to yield 0.36 g of Form A2 crystals.

Basic crystallographic data for the new solid-state acetone solvate Form A2 are represented in Table 3.

The new solid-state acetone solvate Form A2 has characteristic x-ray powder diffraction peaks designated by “2Θ” and expressed in degrees as follows: 5.4±0.2°, 11.3±0.2°, 13.8±0.2°, 17.1±0.2°, 23.3±0.2° and 27.1±0.2°.

### Example 6

Crude pantoprazole sodium (5.0 g) was dissolved in acetone (50 ml). After cooling to room temperature, the solution was filtered and stirred for 5 hours at the same temperature. The obtained suspension was filtered. The crystals obtained were separated, washed with methyl acetate, and dried at room temperature and atmospheric pressure for 12 hours. Yield: 4.8 g of Form A3 crystals.

The new solid-state acetone solvate Form A3 has characteristic x-ray powder diffraction peaks designated by “2Θ” and expressed in degrees as follows: 5.4±0.2°; 13.8±0.2°; 16.2±0.2° and 26.2±0.2°.

### Example 7

Crude pantoprazole sodium (5.0 g) was dissolved in acetone (50 ml) and 2.5 ml of water was added. After cooling to room temperature, the solution was filtered and stirred for 5 hours at the same temperature. The obtained suspension was filtered. The separated crystals were washed with acetone and dried at room temperature and atmospheric pressure for 24 hours. Yield: 4.9 g of Form A4 crystals.

The new solid-state acetone solvate Form A4 has characteristic x-ray powder diffraction peaks designated by “2Θ” and expressed in degrees as follows: 5.6±0.2°, 15.4±0.2°, 16.8±0.2°; 17.3±0.2°; 19.6±0.2°; 20.9±0.2°; 24.5±0.2°; 30.1±0.2° and 30.6±0.2°.

### Example 8

Pantoprazole sodium (0.10 g) was dissolved in methyl acetate (5 ml). After cooling to room temperature, the solution was filtered and left at the same temperature for 24 hours. The crystals obtained were separated by suction and dried at room temperature and atmospheric pressure for 18 hours to yield 0.036 g of Form B1 crystals.

Basic crystallographic data for the new solid-state methyl acetate solvate Form B1 are represented in Table 4.

The new solid-state methyl acetate solvate Form B1 has characteristic x-ray powder diffraction peaks designated by “2Θ” and expressed in degrees as follows: 5.3±0.2°, 9.9±0.2°, 11.1±0.2°, 13.3±0.2°, 15.8±0.2°, 19.8±0.2°, 21.4±0.2°, 26.1±0.2°, 26.5±0.2°, 28.9±0.2°and 30.5±0.2°.

### Example 9

Pantoprazole sodium (5.0 g) was dissolved in methyl acetate (50 ml). After cooling to room temperature, the solution was filtered and stirred for 5 hours at the same temperature. The obtained suspension was filtered. The separated crystals were washed with methyl acetate and dried at room temperature and atmospheric pressure for 10 hours. Yield: 4.7 g of Form B2 crystals.

The new solid-state methyl acetate solvate Form B2 has values of characteristic x-ray powder diffraction peaks designated by “2Θ” and expressed in degrees as follows: 5.4±0.2°, 11.2±0.2°, 13.3±0.2°, 16.8±0.2°, 20.5±0.2°, 22.4±0.2° and 26.6±0.2°.

### Example 10

Crude pantoprazole sodium (5.0 g) was dissolved in methyl acetate (50 ml). After cooling to room temperature, the solution was filtered and stirred for 5 hours at the same temperature. The obtained suspension was filtered. The separated crystals were washed with methyl acetate and dried at room temperature and atmospheric pressure for 5 hours. Yield: 4.4 g of Form B2 crystals.

The x-ray powder pattern of the thus obtained sample corresponds to the x-ray powder pattern of the new solid-state methyl acetate solvate Form B2 product obtained in Example 9.

### Example 11

Pantoprazole sodium (5.0 g) was dissolved in methyl acetate (50 ml) and 2.5 ml of water was added. After cooling to room temperature, the solution was filtered and mixed for 5 hours at the same temperature. The obtained suspension was filtered, and the separated crystals were washed with methyl acetate and dried at room temperature and atmospheric pressure for 10 hours. Yield: 4.6 g of Form B3 crystals.

The new solid-state methyl acetate solvate Form B3 has characteristic x-ray powder diffraction peaks designated by “2Θ” and expressed in degrees as follows: 5.5±0.2°, 9.5±0.2°, 11.9±0.2°, 15.3±0.2°, 19.2±0.2°, 23.9±0.2° and 33.0±0.2°.

### Example 12

Crude pantoprazole sodium (5.0 g) was dissolved in methyl acetate (50 ml) and 2.5 ml of water was added. After cooling to room temperature, the solution was filtered and mixed for 5 hours at the same temperature. The obtained suspension was filtered. The separated crystals were washed with methyl acetate and dried at room temperature and atmospheric pressure for 16 hour. Yield: 4.4 g of Form B3 crystals.

The x-ray powder pattern of the thus obtained sample corresponds to the x-ray powder pattern of the solid-state methyl acetate solvate Form B3 product obtained in Example 11.

### Example 13

Pantoprazole sodium (0.50 g) was dissolved in methyl ethyl ketone (10 ml). After cooling to room temperature, the solution was filtered and left at the same temperature for 24 hours. The crystals obtained were separated by suction and dried at room temperature and atmospheric pressure for 20 hours to yield 0.43 g of Form C1 crystals.

Basic crystallographic data for the new solid-state methyl ethyl ketone solvate Form C1 are represented in Table 5.

The new solid-state methyl ethyl ketone solvate Form C1 has characteristic x-ray powder diffraction peaks designated by “2Θ” and expressed in degrees as follows: 5.5±0.2°, 10.4±0.2°, 10.9±0.2°, 19.2±0.2°, 20.5±0.2°, 21.4±0.2°, 24.6±0.2°, 29.7±0.2°, 33.0±0.2°and 33.9±0.2°.

### Example 14

Pantoprazole sodium (5.0 g) was dissolved in methyl ethyl ketone (50 ml) and 2.5 ml of water was added. After cooling to room temperature, the solution was filtered and mixed for 5 hours at the same temperature. The obtained suspension was filtered, and the separated crystals were washed with methyl ethyl ketone and dried at room temperature and atmospheric pressure for 24 hours. Yield: 4.9 g of Form C1 crystals.

The x-ray powder pattern of the thus obtained sample corresponds to the x-ray powder pattern of the solid-state methyl ethyl ketone solvate Form C1 product obtained in Example 13.

### Example 15

Pantoprazole sodium (5.0 g) was dissolved in methyl ethyl ketone (50 ml). After cooling to room temperature, solution was filtered and mixed for 5 hours at the same temperature. The obtained suspension was filtered. The separated crystals were washed with methyl ethyl ketone and dried at room temperature and atmospheric pressure for 6 hours. Yield: 4.7 g of Form C2 crystals.

The new solid-state methyl ethyl ketone solvate Form C2 has characteristic x-ray powder diffraction peaks designated by “2Θ” and expressed in degrees as follows: 5.4±0.2°, 10.7±0.2°, 12.3±0.2°, 15.8±0.2°, 16.7±0.2°, 20.1±0.2° and 22.5±0.2°.

### Example 16

Pantoprazole sodium (0.5 g) was dissolved in diethyl ketone (15 ml). After cooling to room temperature the solution was filtered. The obtained solution was left at the same temperature for 24 hours. Thus obtained crystals were separated by suction and dried at room temperature and atmospheric pressure for 10 hours to yield 0.38 g of Form D1 crystals.

Basic crystallographic data for the new solid-state diethyl ketone solvate Form D1 are represented in Table 6.

The new solid-state diethyl ketone solvate Form D1 has characteristic x-ray powder diffraction peaks designated by “2Θ” and expressed in degrees as follows: 5.2±0.2°, 10.4±0.2°, 12.3±0.2°, 13.1±0.2°, 15.1±0.2°, 15.8±0.2°, and 25.0±0.2°.

### Example 17

Crude pantoprazole sodium (5.0 g) was dissolved in diethyl ketone (50 ml). After cooling to room temperature, the solution was filtered and then stirred for 6 hours. The obtained suspension was filtered. The separated crystals were washed with diethyl ketone and dried at room temperature and atmospheric pressure for 8 hours. Yield: 2.8 g of Form D1 crystals.

The x-ray powder pattern of the thus obtained sample corresponds to the x-ray powder pattern of the new solid-state diethyl ketone solvate Form D1 product obtained in Example 15.

### Example 18

2.3 g of Form A3 pantoprazole sodium aqua complex, prepared according to Example 6, was dried at 60° C. under a vacuum of 5 mbar for 3 hours to yield 2.0 g of Form E1.

The desolvated Form E1 has characteristic x-ray powder diffraction peaks designated by “2Θ” and expressed in degrees as follows: 5.4±0.2°, 11.6±0.2°, 12.4±0.2°, 13.6±0.2°, 16.0±0.2°, 23.3±0.2° and 28.7±0.2°.

### Example 19

2.4 g of Form A4 pantoprazole sodium aqua complex, prepared according to Example 7, was dried at 60° C. and under a vacuum of 10 mbar for 5 hours to yield 2.0 g of Form E1.

The x-ray powder pattern of the thus obtained sample corresponds to the x-ray powder pattern of the new solid-state desolvated Form E1 product obtained in Example 18.

### Example 20

2.3 g of Form B2 of pantoprazole sodium aqua complex, prepared according to Example 9 were dried at 80° C. and under vacuum of 5 mbar for 1 hour yielding 1.9 g of form E1.

The x-ray powder pattern of the thus obtained sample corresponds to the x-ray powder pattern of the new solid-state desolvated Form E1 product obtained in Example 18.

### Example 21

2.8 g of Form B3 of pantoprazole sodium aqua complex, prepared according to Example 11, was dried at 120° C. and under vacuum of 2 mbar for 2 hours yielding 2.4 g of Form E1.

The x-ray powder pattern of the thus obtained sample corresponds to the x-ray powder pattern of the new solid-state desolvated Form E1 product obtained in Example 18.

### Example 22

2.8 g of Form B3 pantoprazole sodium aqua complex, prepared according to Example 12, was dried at 60° C. and under vacuum of 5 mbar for 3 hours to yield 2.4 g of Form E1.

The x-ray powder pattern of the thus obtained sample corresponded to the x-ray powder pattern of the new solid-state desolvated Form E1 product obtained in Example 18.

### Example 23

3.3 g of Form C2 of pantoprazole sodium aqua complex, prepared according to Example 14, was dried at 50° C. and under vacuum of 5 mbar for 4 hours to yield 2.3 g of Form E1.

The x-ray powder pattern of the thus obtained sample corresponded to the x-ray powder pattern of the new solid-state desolvated Form E1 product obtained in Example 18.

### Example 24

2.9 g of Form C2 of pantoprazole sodium aqua complex, prepared according to Example 15, was dried at 25° C. and under vacuum of 1 mbar for 6 hours to yield 2.5 g of Form E1.

The x-ray powder pattern of the thus obtained sample corresponded to the x-ray powder pattern of the new solid-state desolvated Form E1 product obtained in Example 18.

### Example 25

1.4 g of Form D1 pantoprazole sodium aqua complex, prepared according to Example 16, was dried at 60° C. and under vacuum of 5 mbar for 5 hour to yield 1.2 g of Form E1.

The x-ray powder pattern of the thus obtained sample corresponded to the x-ray powder pattern of the new solid-state desolvated Form E1 obtained in Example 18.

1. A solid-state form of pantoprazole, comprising a sodium aqua complex chosen from:
(i) organic solvent free hexacoordinated octahedral Form N, characterized by the orthorhombic space group P bca, and unit cell parameters comprising: crystal axis lengths of a=17.10(2) Å, b=13.49 (1) Å, c=33.15(2) Å and angles between the crystal axes of α=β=γ=90°;
(ii) acetone solvate hexacoordinated octahedral Form A1, characterized by the monoclinic space group P2₁, and displaying unit cell parameters comprising: crystal axis lengths of a=13.58(2) Å, b=10.63(1) Å, c=15.72 (2) Å and an angle between the crystal axes off β=90.5(1)°;
(iii) acetone solvate pentacoordinated square pyramidal Form A2, characterized by the monoclinic space group P2₁/a, and displaying unit cell parameters comprising: crystal axis lengths of a=13.18(1) Å, b=10.27(1) Å, c=17.28 (2) Å and an angle between the crystal axes off β=109.1(1)°;
(iv) acetone solvate Form A3, having characteristic x-ray powder diffraction peaks, designated by 2Θand expressed in degrees, at: 5.4±0.2°; 11.2±0.2°; 16.9±0.2°; 17.6±0.2°° 19.5±0.2° and 26.2±0.2°.;
(v) acetone solvate Form A4, having characteristic x-ray powder diffraction peaks, designated by 2Θ and expressed in degrees, at: 5.6±0.2°, 15.4±0.2°, 16.8±0.2°; 17.3±0.2°; 19.6±0.2°; 20.9±0.2±; 24.5±0.2°; 30.1±0.2°and 30.6±0.2°;
(vi) methyl acetate solvate hexacoordinated octahedral Form B1, characterized by the monoclinic space group P2₁/a, and displaying unit cell parameters comprising: crystal axis lengths of a=13.31(1) Å, b=10.47(1) Å, c=17.68(2) Å and an angle between the crystal axes off β=109.9(1)°;
(vii) methyl acetate solvate Form B2, having characteristic x-ray powder diffraction peaks, designated by 2Θ and expressed in degrees, at: 5.4±0.2°, 11.2±0.2°, 13.3±0.2°, 16.8±0.2°, 20.5±0.2°, 22.4±0.2° and 26.6±0.2°;
(viii) methyl acetate solvate Form B3, having characteristic x-ray powder diffraction peaks, designated by 2Θ and expressed in degrees, at: 5.5±0.2°, 9.5±0.2°, 11.9±0.2°, 15.3±0.2°, 19.2±0.2°, 23.9±0.2°and 33.0±0.2°;
(ix) methyl ethyl ketone solvate hexacoordinated octahedral Form C1, characterized by the monoclinic space group P2₁/a, and displaying unit cell parameters comprising: crystal axis lengths of a=13.51(1) Å, b=10.66(1) Å, c=16.16(2) Å and an angle between the crystal axes of β=92.3(1)°;
(x) methyl ethyl ketone solvate Form C2, having characteristic x-ray powder diffraction peaks, designated by 2Θ and expressed in degrees, at: 5.4±0.2°, 10.7±0.2°, 12.3±0.2°, 15.8±0.2°, 16.7±0.2°, 20.1±0.2°and 22.5±0.2°;
(xi) diethyl ketone solvate hexacoordinated octahedral Form D1, characterized by the monoclinic space group P2₁/a, and displaying unit cell parameters comprising: crystal axis lengths of a=13.42(1) Å, b=10.85(1) Å, c=17.36(2) Å and an angle between the crystal axes off β=102.5(1)°; and
(xii) desolvated Form E1, having characteristic x-ray powder diffraction peaks, designated by 2Θ and expressed in degrees, at: 5.4±0.2°, 11.6±0.2°, 12.4±0.2°, 13.6±0.2°, 16.0±0.2°, 23.3±0.2°and 28.7±0.2°.

2. The solid-state form of pantoprazole of claim 1, wherein
Form N has characteristic x-ray powder diffraction peaks, designated by 2Θ and expressed in degrees, at: 5.3±0.2°, 13.1±0.2°, 16.9±0.2°, 20.5±0.2°, 21.6±0.2°and 25.1±0.2°;
Form A1 has characteristic x-ray powder diffraction peaks, designated by 2Θ and expressed in degrees, at: 5.6±0.2°, 11.9±0.2°, 12.9±0.2°, 13.8±0.2°, 15.4±0.2°, 16.4±0.2°and 26.1±0.2°;
Form A2 has characteristic x-ray powder diffraction peaks, designated by 2Θ and expressed in degrees, at: 5.4±0.2°, 11.3±0.2°, 13.8±0.2°, 17.1±0.2°, 23.3±0.2° and 27.1±0.2°;
Form B1 has characteristic x-ray powder diffraction peaks, designated by 2Θ and expressed in degrees, at: 5.3±0.2°, 9.9±0.2°, 11.1±0.2°, 13.3±0.2°, 15.8±0.2°, 19.8±0.2°, 21.4±0.2°, 26.1±0.2°, 26.5±0.2°, 28.9±0.2°and 30.5±0.2°;
Form C1 has characteristic x-ray powder diffraction peaks, designated by 2Θ and expressed in degrees, at: 5.5±0.2°, 10.4±0.2°, 10.9±0.2°, 19.2±0.2°, 20.5±0.2°, 21.4±0.2°, 24.6±0.2°, 29.7±0.2°, 33.0±0.2° and 33.9±0.2°; and
Form D1 has characteristic x-ray powder diffraction peaks, designated by 2Θ and expressed in degrees, at: 5.2±0.2°, 10.4±0.2°, 12.3±0.2°, 13.1±0.2°, 15.1±0.2°, 15.8±0.2°and 25.0±0.2°.

3. The solid-state form of pantoprazole of claim 1, chosen from Form N and Form E1.

4. The solid-state form of pantoprazole of claim 3, having a solid-state purity greater than 95.0%.

5. The solid-state form of pantoprazole of claim 3, having a solid-state purity greater than 99.0%.

6. The solid-state form of pantoprazole of claim 3, having a solid-state purity greater than 99.5%.

7. The solid-state form of pantoprazole of claim 3, having a solid-state purity greater than 99.9%.

8. The solid-state form of pantoprazole of claim 3, having a chemical purity of greater than about 98.0%.

9. The solid-state form of pantoprazole of claim 3, having a chemical purity of greater than about 99.0%.

10. The solid-state form of pantoprazole of claim 2 having a chemical purity of greater than about 99.5%.

11. The solid-state form of pantoprazole of claim 2, having a chemical purity of greater than about 99.9%.

12. The solid-state form of pantoprazole of claim 2, wherein the complex is stable under normal storage conditions.

13. The solid-state form of pantoprazole of claim 1, chosen from Forms A1, A2, A3, A4, B1, B2, B3, C1, C2, and D1.

14. The solid-state form of pantoprazole of claim 13, having a chemical purity of greater than about 98.0%.

15. The solid-state form of pantoprazole of claim 13, having a chemical purity of greater than about 99.0%.

16. The solid-state form of pantoprazole of claim 13, having a chemical purity of greater than about 99.5%.

17. The solid-state form of pantoprazole of claim 13, having a chemical purity of greater than about 99.9%.

18. The solid-state form of pantoprazole of claim 13, wherein the complex is stable under normal storage conditions.

19. A process for the preparation of a solid-state form of pantoprazole chosen from Forms N, A4, B3, and C1 of claim 1, comprising:
(i) suspending pantoprazole sodium salt in an organic solvent;
(ii) dissolving the pantoprazole sodium salt in the organic solvent;
(iii) optionally filtering the solution of pantoprazole sodium salt and organic solvent;
(iv) adding water;
(v) crystallizing the solid-state form of pantoprazole;
(vi) isolating the crystals thus obtained; and
(vii) drying the crystals;
wherein
for solid-state Form N, the organic solvent is an aliphatic ester or mixture thereof;
for solid-state Form A4, the organic solvent is acetone;
for solid-state Form B3, the organic solvent is methyl acetate; and
for solid-state Form C1 the organic solvent is methyl ethyl ketone.

20. The process of claim 19, wherein the aliphatic ester is chosen from ethyl acetate, propyl acetate, isopropyl acetate, butyl acetate, sec-butyl acetate and tert-butyl acetate.

21. The process of claim 19, wherein step (ii) comprises heating the suspension of pantoprazole sodium salt and organic solvent to a temperature of from about 30° C. to about reflux.

22. The process of claim 19, wherein the solution of step (iii) is filtered.

23. The process of claim 19, wherein step (iv) comprises adding water in an amount of about 0.1 to about 5% by volume of organic solvent.

24. The process of claim 23, wherein the water is added in an amount of about 2.5% by volume of organic solvent.

25. The process of claim 19, wherein step (v) comprises cooling the solution to from about 70° C. to about −10° C.

26. The process of claim 25, wherein the solution is cooled to about room temperature.

27. The process of claim 19, wherein step (v) comprises crystallizing the complex over a time period of from about 15 minutes to about 24 hours.

28. The process of claim 4, wherein step (vii) comprises drying the crystals at a pressure of from about atmospheric pressure to about 5 mbar.

29. The process of claim 4, wherein step (vii) comprises drying the crystals at a temperature of from about room temperature to about 100° C.

30. The process of claim 4, wherein step (vii) comprises drying the crystals for a period of time of from about 1 hour to about 24 hours.

31. A solid-state form of pantoprazole chosen from Forms N, A4, B1, B3, and C1, prepared by the process of claim 19.

32. A process for the preparation of a solid-state complex chosen from Forms A1, A2, A3, B1, B2, C1, C2 and D1 of claim 1, comprising:
(i) suspending pantoprazole sodium salt in an organic solvent;
(ii) dissolving the pantoprazole sodium salt in the organic solvent;
(iii) optionally filtering the solution of pantoprazole sodium salt and organic solvent;
(iv) crystallizing the solid-state form of pantoprazole;
(v) isolating the crystals thus obtained; and
(vi) drying the crystals,
wherein
for solid-state Form A1, the organic solvent is acetone;
for solid-state Form A2, the organic solvent is acetone;
for solid-state Form A3, the organic solvent is acetone;
for solid-state Form B1, the organic solvent is methyl acetate;
for solid-state Form B2, the organic solvent is methyl acetate;
for solid-state Form C1, the organic solvent is methyl ethyl ketone;
for solid-state Form C2, the organic solvent is methyl ethyl ketone; and
for solid-state Form D1, the organic solvent is diethyl ketone.

33. The process of claim 32, wherein step (ii) comprises heating the suspension of pantoprazole sodium salt and organic solvent to a temperature of from about 30° C. to about reflux.

34. The process of claim 32, wherein the solution of step (iii) is filtered.

35. The process of claim 32, wherein step (iv) comprises cooling the solution to from about 70 ° C. to about −10° C.

36. The process of claim 35, wherein the solution is cooled to about room temperature.

37. The process of claim 32, wherein step (iv) comprises crystallizing the complex over a time period of from about 15 minutes to about 24 hours.

38. The process of claim 32, wherein step (vi) comprises drying the crystals at pressure of about atmospheric pressure.

39. The process of claim 32, wherein step (vi) comprises drying the crystals at a temperature of about room temperature.

40. The process of claim 32, wherein step (vi) comprises drying the crystals for a period of time of from about 1 hour to about 24 hours.

41. A solid-state form of pantoprazole chosen from Forms A1, A2, A3, B1, B2, C1, C2 and D1, prepared by the process of claim 32.

42. A process for the preparation of the solid-state Form E1 of pantoprazole according to claim 1, comprising drying solvates of pantoprazole sodium aqua complexes for a period of time sufficient to obtain the desolvated Form E1 complex.

43. The process of claim 42, wherein the drying is conducted at temperatures of from about 20° C. to about 120° C.

44. The process of claim 43, wherein the drying is conducted at a temperature of about 60° C.

45. The process of claim 42, wherein the drying is conducted at a pressure of from about 1 mbar to about 10 mbar.

46. The process of claim 45, wherein the drying is conducted at a pressure of about 5 mbar.

47. The process of claim 42, wherein the drying is conducted for a time period of from about 1 hour to about 6 hours.

48. The process of claim 47, wherein the drying is conducted for a time period of about 3 hours.

49. Desolvated Form E1 sodium aqua complex of pantoprazole, prepared by the process of claim 42.

50. Use of a solid-state sodium aqua complex of pantoprazole of claim 1 as a raw material for the preparation of solid-state monohydrate and sesquihydrate forms of pantoprazole sodium.

51. Use of a solid-state form of pantoprazole of claim 1 as a raw material for the preparation of pantoprazole hexacoordinated octahedral sodium aqua complexes and pantoprazole pentacoordinated square pyramidal aqua complexes.

52. Use of a solid-state sodium aqua complex of pantoprazole of claim 1 as a raw material for the preparation of pharmaceutically acceptable pantoprazole salts.

53. The use of claim 52, wherein the pharmaceutically acceptable pantoprazole salt is the magnesium salt of pantoprazole.

54. A pharmaceutical composition comprising a solid-state form of pantoprazole of claim 1, and a pharmaceutically acceptable carrier.

55. The composition of claim 54, wherein the solid-state form of pantoprazole is chosen from Form N and Form E1.

56. A method for inhibiting gastric acid secretion and protecting the stomach and intestines of a patient in need of such treatment, comprising administering to the patient a therapeutically effective amount of a solid-state form of pantoprazole of claim 1.

57. A method for inhibiting gastric ulcers in a patient in need of such treatment, comprising administering to the patient a therapeutically effective amount of a solid-state form of pantoprazole of claim 1

58. The method of claim 56, wherein the solid-state sodium aqua complex of pantoprazole is chosen from Form N and Form E1.

59. The method of claim 57, wherein the solid-state sodium aqua complex of pantoprazole is chosen from Form N and Form E1.
